SEQUENCE LISTING (24 sequences)

SEQ ID NO: 1 - DNA, 2682 bp, Homo sapiens
gcggccgcgtcgacctaagtccctttttcttgagagagtgtggctctgtcaccaaggctggagtgcaggggcacaatctcagctcactgcaacctccaccttgcgtgtctcagcctcctgagcagctgggaccgcgggagtgtgccaccacacccagctaatttttgtatttttttagtggagatggggtgtcaccatgttggccaggctggtctcaaactctcggcctcagcctcccaaagtgctgggattataggtgtgagccaccacacccagcctctatatcccttttatcggtggacccaacaatcttctgtagacagaagactgtgtgaataagccaatggtcccaagagagcacgcatcctaatgagaacggagacagataataaagagggaaacaagtaaaccaactatcaatgattaggagcgtcagatagagccaggtgcagcctggacaacatgatgagatccccatctctaaaagtataaaataaattggctggtcatgatggtgcacacctgtggccccagcttctcagtaggctgaggtgggaggattgcttgagcctgggaggtcaagcctacagtgagccatgatctcaccatgcactccagcctagacaacagggcaaggccctctaaaaaaaaaaatgaaaagatagcatgctttggccggatgtggtggctcacgcctgtaatcccagcacttcggggggttgaggcgggcagatcactgaggtcaggagtacgagatcagcctggccagcacggtgaaaccccgtctctgctgaaggtgcaaaattgactgggcgtggtggctcatgcctgtagtcccagctactctggaggctgaggcgggagaatcgcttgaacccgggaggcggaggttgtggtgagctgagattgcgccattgcactccagcctgggcaacaagagcgaaactccgtctcaaaaaaggatagcatgcttcataaacggtgaaatgacatgcaaaggccatagttgtcaagcaccagtaacaccagagagtggcctgtctttccttcaagcagaccagagaggaccgttctgctggacgcctacagtgaacatctggcagcggccctgtgcaccaatccaaacctgatagagctgtctctgtaccgaaatgccctgggcagccggggggtgaagctgctctgtcaaggactcagacaccccaactgcaaacttcagaacctgagcactttgggaggcccagatggggaggatcacttgacccaggagttcaagaccagcctggccaacatggtgaaaccccatctctactaaaaataccaaaatgagccaggcatggtggcacacgtctgtaagcccagctactcaggaggccaaggcaggaggattgcttcaacccaggaggcagaggttgtggctgaagaggtgccgcatctccagctcagcctgcgaggacctctctgcagctctcatagccaataagaatttgacaaggatggatctcagtggcaacggcgttggattcccaggcatgatgctgctttgcgagggcctgcggcatccccagtgcaggctgcagatgattcagttgaggaagtgtcagctggagtccggggcttgtcaggagatggcttctgtgctcggcaccaacccacatctggttgagttggacctgacaggaaatgcactggaggatttgggcctgaggttactatgccagggactgaggcacccagtctgcagactacggactttgtggctgaagatctgccgcctcactgctgctgcctgtgacgagctggcctcaactctcagtgtgaaccagagcctgagagagctggacctgagcctgaatgagctgggggacctcggggtgctgctgctgtgtgagggcctcaggcatcccacgtgcaagctccagaccctgcggttgggcatctgccggctgggctctgccgcctgtgagggtctttctgtggtgctccaggccaaccacaacctccgggagctggacttgagtttcaacgacctgggagactggggcctgtggttgctggctgaggggctgcaacatcccgcctgcagactccagaaactgtggctggatagctgtggcctcacagccaaggcttgtgagaatctttacttcaccctggggatcaaccagaccttgaccgacctttacctgaccaacaacgccctaggggacacaggtgtccgactgctttgcaagcggctgagccatcctggctgcaaactccgagtcctctggttatttgggatggacctgaataaaatgacccacagtaggttggcagcgcttcgagtaacaaaaccttatttggacattggctgctgaatggtcctatctgctggctctcccctgagatctggacagaggaagatgggagggtgctcatcacccccccagcataatgatcagcctccttcctagagacagactcatgcagattgagatcaaaagtccctctgcttgggatcaaattaatgtttgacagagctggccaggcgtggtggctcatgtatgtaatcctagcacttcgagaggccgaggcaggtggatcacgaggtcaggagtttgagattagcctggccaagatggtgaaaccctgtctctactaaaaataaaaaaaaattagccaggaaaaaaaaaaaaaaaa

SEQ ID NO: 2 - protein, 344 aa, Homo sapiens
MSQAWWHTSVSPATQEAKAGGLLQPRRQRLWLKRCRISSSACEDLSAALIANKNLTRMDLSGNGVGFPGMMLLCEGLRHPQCRLQMIQLRKCQLESGACQEMASVLGTNPHLVELDLTGNALEDLGLRLLCQGLRHPVCRLRTLWLKICRLTAAACDELASTLSVNQSLRELDLSLNELGDLGVLLLCEGLRHPTCKLQTLRLGICRLGSAACEGLSVVLQANHNLRELDLSFNDLGDWGLWLLAEGLQHPACRLQKLWLDSCGLTAKACENLYFTLGINQTLTDLYLTNNALGDTGVRLLCKRLSHPGCKLRVLWLFGMDLNKMTHSRLAALRVTKPYLDIGC

SEQ ID NO: 3 - DNA, 2494 bp, Homo sapiens
agtccctttttcttgagagagtgtggctctgtcaccaaggctggagtgcaggggcacaatctcagctcactgcaacctccaccttgcgtgtctcagcctcctgagcagctgggaccgcgggagtgtgccaccacacccagctaatttttgtatttttttagtggagatggggtgtcaccatgttggccaggctggtctcaaactctcggcctcagcctcccaaagtgctgggattataggtgtgagccaccacacccagcctctatatcccttttatcggtggacccaacaatcttctgtagacagaagactgtgtgaataagccaatggtcccaagagagcacgcatcctaatgagaacggagacagataataaagagggaaacaagtaaaccaactatcaatgattaggagcgtcagatagagccaggtgcagcctggacaacatgatgagatccccatctctaaaagtataaaataaattggctggtcatgatggtgcacacctgtggccccagcttctcagtaggctgaggtgggaggattgcttgagcctgggaggtcaagcctacagtgagccatgatctcaccatgcactccagcctagacaacagggcaaggccctctaaaaaaaaaaatgaaaagatagcatgctttggccggatgtggtggctcacgcctgtaatcccagcacttcggggggttgaggcgggcagatcactgaggtcaggagtacgagatcagcctggccagcacggtgaaaccccgtctctgctgaaggtgcaaaattgactgggcgtggtggctcatgcctgtagtcccagctactctggaggctgaggcgggagaatcgcttgaacccgggaggcggaggttgtggtgagctgagattgcgccattgcactccagcctgggcaacaagagcgaaactccgtctcaaaaaaggatagcatgcttcataaacggtgaaatgacatgcaaaggccatagttgtcaagcaccagtaacaccagagagtggcctgtctttccttcaagcagaccagagaggaccgttctgctggacgcctacagtgaacatctggcagcggccctgtgcaccaatccaaacctgatagagctgtctctgtaccgaaatgccctgggcagccggggggtgaagctgctctgtcaaggactcagacaccccaactgcaaacttcagaacctgagcactttgggaggcccagatggggaggatcacttgacccaggagttcaagaccagcctggccaacatggtgaaaccccatctctactaaaaataccaaaatgagccaggcatggtggcacacgtctgtaagcccagctactcaggaggccaaggcaggaggattgcttcaacccaggaggcagaggttgtggctgaagaggtgccgcatctccagctcagcctgcgaggacctctctgcagctctcatagccaataagaatttgacaaggatggatctcagtggcaacggcgttggattcccaggcatgatgctgctttgcgagggcctgcggcatccccagtgcaggctgcagatgattcagttgaggaagtgtcagctggagtccggggcttgtcaggagatggcttctgtgctcggcaccaacccacatctggttgagttggacctgacaggaaatgcactggaggatttgggcctgaggttactatgccagggactgaggcacccagtctgcagactacggactttgtggctgaagatctgccgcctcactgctgctgcctgtgacgagctggcctcaactctcagtgtgaaccagagcctgagagagctggacctgagcctgaatgagctgggggacctcggggtgctgctgctgtgtgagggcctcaggcatcccacgtgcaagctccagaccctgcggctggatagctgtggcctcacagccaaggcttgtgagaatctttacttcaccctggggatcaaccagaccttgaccgacctttacctgaccaacaacgccctaggggacacaggtgtccgactgctttgcaagcggctgagccatcctggctgcaaactccgagtcctctggttatttgggatggacctgaataaaatgacccacagtaggttggcagcgcttcgagtaacaaaaccttatttggacattggctgctgaatggtcctatctgctggctctcccctgagatctggacagaggaagatgggagggtgctcatcacccccccagcataatgatcagcctccttcctagagacagactcatgcagattgagatcaaaagtccctctgcttgggatcaaattaatgtttgacagagctggccaggcgtggtggctcatgtatgtaatcctagcacttcgagaggccgaggcaggtggatcacgaggtcaggagtttgagattagcctggccaagatggtgaaaccctgtctctactaaaaataaaaaaaaattagccaggaaaaaaaaaaaaaaaa

SEQ ID NO: 4 - protein, 287 aa, Homo sapiens
MSQAWWHTSVSPATQEAKAGGLLQPRRQRLWLKRCRISSSACEDLSAALIANKNLTRMDLSGNGVGFPGMMLLCEGLRHPQCRLQMIQLRKCQLESGACQEMASVLGTNPHLVELDLTGNALEDLGLRLLCQGLRHPVCRLRTLWLKICRLTAAACDELASTLSVNQSLRELDLSLNELGDLGVLLLCEGLRHPTCKLQTLRLDSCGLTAKACENLYFTLGINQTLTDLYLTNNALGDTGVRLLCKRLSHPGCKLRVLWLFGMDLNKMTHSRLAALRVTKPYLDIGC

SEQ ID NO: 5 - DNA, 2511 bp, Homo sapiens
gcggccgcgtcgacctaagtccctttttcttgagagagtgtggctctgtcaccaaggctggagtgcaggggcacaatctcagctcactgcaacctccaccttgcgtgtctcagcctcctgagcagctgggaccgcgggagtgtgccaccacacccagctaatttttgtatttttttagtggagatggggtgtcaccatgttggccaggctggtctcaaactctcggcctcagcctcccaaagtgctgggattataggtgtgagccaccacacccagcctctatatcccttttatcggtggacccaacaatcttctgtagacagaagactgtgtgaataagccaatggtcccaagagagcacgcatcctaatgagaacggagacagataataaagagggaaacaagtaaaccaactatcaatgattaggagcgtcagatagagccaggtgcagcctggacaacatgatgagatccccatctctaaaagtataaaataaattggctggtcatgatggtgcacacctgtggccccagcttctcagtaggctgaggtgggaggattgcttgagcctgggaggtcaagcctacagtgagccatgatctcaccatgcactccagcctagacaacagggcaaggccctctaaaaaaaaaaatgaaaagatagcatgctttggccggatgtggtggctcacgcctgtaatcccagcacttcggggggttgaggcgggcagatcactgaggtcaggagtacgagatcagcctggccagcacggtgaaaccccgtctctgctgaaggtgcaaaattgactgggcgtggtggctcatgcctgtagtcccagctactctggaggctgaggcgggagaatcgcttgaacccgggaggcggaggttgtggtgagctgagattgcgccattgcactccagcctgggcaacaagagcgaaactccgtctcaaaaaaggatagcatgcttcataaacggtgaaatgacatgcaaaggccatagttgtcaagcaccagtaacaccagagagtggcctgtctttccttcaagcagaccagagaggaccgttctgctggacgcctacagtgaacatctggcagcggccctgtgcaccaatccaaacctgatagagctgtctctgtaccgaaatgccctgggcagccggggggtgaagctgctctgtcaaggactcagacaccccaactgcaaacttcagaacctgagcactttgggaggcccagatggggaggatcacttgacccaggagttcaagaccagcctggccaacatggtgaaaccccatctctactaaaaataccaaaatgagccaggcatggtggcacacgtctgtaagcccagctactcaggaggccaaggcaggaggattgcttcaacccaggaggcagaggttgtggctgaagaggtgccgcatctccagctcagcctgcgaggacctctctgcagctctcatagccaataagaatttgacaaggatggatctcagtggcaacggcgttggattcccaggcatgatgctgctttgcgagggcctgcggcatccccagtgcaggctgcagatgattcagttgaggaagtgtcagctggagtccggggcttgtcaggagatggcttctgtgctcggcaccaacccacatctggttgagttggacctgacaggaaatgcactggaggatttgggcctgaggttactatgccagggactgaggcacccagtctgcagactacggactttgtggctgaagatctgccgcctcactgctgctgcctgtgacgagctggcctcaactctcagtgtgaaccagagcctgagagagctggacctgagcctgaatgagctgggggacctcggggtgctgctgctgtgtgagggcctcaggcatcccacgtgcaagctccagaccctgcggttgggcatctgccggctgggctctgccgcctgtgagggtctttctgtggtgctccaggccaaccacaacctccgggagctggacttgagtttcaacgacctgggagactggggcctgtggttgctggctgaggggctgcaacatcccgcctgcagactccagaaactgtggttatttgggatggacctgaataaaatgacccacagtaggttggcagcgcttcgagtaacaaaaccttatttggacattggctgctgaatggtcctatctgctggctctcccctgagatctggacagaggaagatgggagggtgctcatcacccccccagcataatgatcagcctccttcctagagacagactcatgcagattgagatcaaaagtccctctgcttgggatcaaattaatgtttgacagagctggccaggcgtggtggctcatgtatgtaatcctagcacttcgagaggccgaggcaggtggatcacgaggtcaggagtttgagattagcctggccaagatggtgaaaccctgtctctactaaaaataaaaaaaaattagccaggaaaaaaaaaaaaaaaa

SEQ ID NO: 6 - protein, 287 aa, Homo sapiens
MSQAWWHTSVSPATQEAKAGGLLQPRRQRLWLKRCRISSSACEDLSAALIANKNLTRMDLSGNGVGFPGMMLLCEGLRHPQCRLQMIQLRKCQLESGACQEMASVLGTNPHLVELDLTGNALEDLGLRLLCQGLRHPVCRLRTLWLKICRLTAAACDELASTLSVNQSLRELDLSLNELGDLGVLLLCEGLRHPTCKLQTLRLGICRLGSAACEGLSVVLQANHNLRELDLSFNDLGDWGLWLLAEGLQHPACRLQKLWLFGMDLNKMTHSRLAALRVTKPYLDIGC

SEQ ID NO: 7 - DNA, 24 bp, Artificial
  synthetic oligonucleotide
gataagaccattcagcagccaatg

SEQ ID NO: 8 - DNA, 24 bp, Artificial
  synthetic oligonucleotide
agtgtgaaccagagcctgagagag

SEQ ID NO: 9 - DNA, 20 bp, Artificial
  synthetic oligonucleotide
cgctgcgctggtcgtcgaca

SEQ ID NO: 10 - DNA, 20 bp, Artificial
  synthetic oligonucleotide
gtcacgcacgatttcccgct

SEQ ID NO: 11 - DNA, 6 bp, Artificial
  Consensus Sequence
aataaa

SEQ ID NO: 12 - protein, 8 aa, Artificial
  Consensus Sequence
CXLTXXXC

SEQ ID NO: 13 - protein, 7 aa, Artificial
  Consensus sequence
NXLGDXG

SEQ ID NO: 14 - protein, 315 aa, Homo sapiens
RLEKLQLEYCSLSAASCEPLASVLRAKPDFKELTVSNNDINEAGVRVLCQGLKDSPCQLEALKLESCGVTSDNCRDLCGIVASKASLRELALGSNKLGDVGMAELCPGLLHPSSRLRTLWIWECGITAKGCGDLCRVLRAKESLKELSLAGNELGDEGARLLCETLLEPGCQLESLWVKSCSFTAACCSHFSSVLAQNRFLLELQISNNRLEDAGVRELCQGLGQPGSVLRVLWLADCDVSDSSCSSLAATLLANHSLRELDLSNNCLGDAGILQLVESVSEPGCLLEQLVLYDIYWSEEMEDRLQALEKDKPSL

SEQ ID NO: 15 - protein, 28 aa, Homo sapiens
RRQRLWLKRCRISSSACEDLSAALIANK

SEQ ID NO: 16 - protein, 28 aa, Homo sapiens
RLQMIQLRKCQLESGACQEMASVLGTNP

SEQ ID NO: 17 - protein, 28 aa, Homo sapiens
RLRTLWLKICRLTAAACDELASTLSVNQ

SEQ ID NO: 18 - protein, 28 aa, Homo sapiens
KLQTLRLGICRLGSAACEGLSVVLQANH

SEQ ID NO: 19 - protein, 28 aa, Homo sapiens
RLQKLWLDSCGLTAKACENLYFTLGINQ

SEQ ID NO: 20 - protein, 29 aa, Homo sapiens
NLTRMDLSGNGVGFPGMMLLCEGLRHPQC

SEQ ID NO: 21 - protein, 29 aa, Homo sapiens
HLVELDLTGNALEDLGLRLLCQGLRHPVC

SEQ ID NO: 22 - protein, 29 aa, Homo sapiens
SLRELDLSLNELGDLGVLLLCEGLRHPTC

SEQ ID NO: 23 - protein, 29 aa, Homo sapiens
NLRELDLSFNDLGDWGLWLLAEGLQHPAC

SEQ ID NO: 24 - protein, 29 aa, Homo sapiens
TLTDLYLTNNALGDTGRRLLCKRLSHPGC

(19) United States
(12) Patent Application Publication
(10) Pub. No.: US 2001/0029033 A1
(43) Pub. Date: Oct. 11, 2001

(54) Novel gene rno upregulated in leukemia cells by nitric oxide and DMSO

(76) Inventors: Paul J. Shami, Salt Lake City, UT (US); Charles J. Parker, Salt Lake City, UT (US)

Correspondence Address: MADSON & METCALF, GATEWAY TOWER WEST, SUITE 900, 15 WEST SOUTH TEMPLE, SALT LAKE CITY, UT, 84101

(21) Appl. No.: 09/799,983

(22) Filed: Mar. 6, 2001

Related U.S. Application Data
(60) Provisional application No. 60/186,971, filed on Mar. 6, 2000.

(51) Int. Cl. C12Q 1/68; G01N 33/574; C12P 21/02; C12N 5/06

(52) U.S. Cl. 435/69.1; 435/325; 435/7.23; 435/6

(57) ABSTRACT

The present invention relates to a novel human gene which has been shown to induce differentiation of cancer cells. The gene is regulated by NO and is named rno. Three isoforms of rno have been isolated and purified, rno-1 (SEQ ID NO: 1), rno-2 (SEQ ID NO: 3), and rno-3 (SEQ ID NO: 5). The rno isoforms each code for a separate amino acid sequence (SEQ ID NOS: 2, 4, & 5 ). The present invention also provides recombinant vectors comprising nucleic acid molecules that code for the rno gene products. In certain embodiments, these recombinant vectors are plasmids. In certain embodiments, these recombinant vectors are prokaryotic or eukaryotic expression vectors. In certain especially preferred embodiments, the nucleic acid coding for the rno gene products are operably linked to a heterologous promoter. The present invention further provides host cells comprising a nucleic acid that codes for the rno gene products (SEQ ID NOS: 2, 4, & 5). The gene products have been shown to induce AML cell differentiation and apoptosis.

### RELATED APPLICATIONS

This application is related to and claims the benefit of United States Provisional Application Serial No. 60/186,971 of Paul J. Shami and Charles J. Parker, filed Mar. 6, 2000 and entitled “Novel Gene (norg or no-regulated) Upregulated in Leukemia Cells by Nitric Oxide and DMSO,” which is incorporated herein by this reference.

### BACKGROUND OF THE INVENTION

2. Field of the Invention

The present invention relates to the isolation and characterization of a novel gene which is upregulated by nitric oxide (NO), hereinafter rno. More specifically, the invention relates to the isolation and characterization of a gene relating to the induction of differentiation and apoptosis of leukemia cells by NO.

2. Technical Background

Nitric oxide (NO) is a naturally occurring molecule with critically important functions in the immunological, neurological, and vascular systems. Nathan C. (1992) FASEB Journal, 6:3051-3064. Studies have shown that NO also has pleiotropic effects on both normal and malignant haematopoietic cells. Treatment of CD34⁺ bone marrow cells with NO inhibits haematopoietic cell colony growth with erythroid colonies being more sensitive to the inhibitory effects of NO than myeloid colonies. Shami, P J & Weinberg, J B. (1996) Blood, 87:977-983. It has also been observed that NO inhibits the growth of the human AML cell line, HL-60. Magrinat, G et al. (1992) Blood, 80:1980-1986. Along with growth inhibition, NO induces HL-60 cells to differentiate and develop a monocytic phenotype as evidenced by enhanced expression of CD11b, CD14 and HLA-DR. Magrinat, Get al. (1992) Blood, 80:1980-1986. NO also inhibits the growth of freshly isolated AML cells and upregulates the expression of CD14 in cells with a monocytic phenotype. Shami, P J et al. (1995) Leukemia Res.19:527-533. Furthermore, NO induces apoptosis in HL-60 and U937 cells in a rate of delivery and concentration dependent manner. Shami, P J et al. (1998) Leukemia.12:1461-1466. NO was also found to modulate gene expression in HL-60 cells with expression of TNFα and IL1β being upregulated and expression of c-myc and c-myb being downregulated. Magrinat, G et al. (1992) Blood, 80:1980-1986.

NO has also been shown to modulate protein and gene expression. NO increases expression of both transferrin receptor mRNA and protein in K562 cells. Oria, R et al. (1995) Blood, 85:2962-2966. These observations were extended by showing stabilization of transferrin receptor mRNA in K562 cells treated with NO. Domachowske, J B et al. (1996) Blood, 88:2980-2988. NO also decreases γ-globin and H-ferritin protein expression in the same cell line. Modulation of expression appears to be a consequence of the effects of NO on the affinity of iron regulatory protein-1 for the iron-responsive elements found in the 3′ and 5′ untranslated regions of these genes. Recently, it was reported that molecular targeting of hypoxia-inducible factor-i explains, at least in part, the effects of NO on expression of the human vascular endothelial growth factor (VEGF) gene. Kimura, H et al. (2000) Blood 95:189-197.

The genes associated with NO-induced differentiation and growth inhibition of leukemia cells has not been determined. Accordingly it would be an advancement in the art to provide a gene associated with NO-induced differentiation and growth inhibition of leukemia cells. It would be a further advancement if the nucleic acid sequences could provide additional understanding of how NO effects the pattern of gene expression.

Such nucleic acid sequences and methods are disclosed and claimed herein.

### BRIEF SUMMARY OF THE INVENTION

The present invention relates to isolated and purified genes which are upregulated by nitric oxide and DMSO. These genes are identified as rno. Provided herein are three isoforms of rno. The isoforms are known as rno-1 (SEQ ID NO: 1), rno-2 (SEQ ID NO: 2), and rno-3 (SEQ ID NO: 3). The amino acid sequence of the rno gene products are also provided. The rno proteins are desingated rno-1 (SEQ ID NO: 2), rno-2 (SEQ ID NO: 4), and rno-3 (SEQ ID NO: 6). Also provided are nucleic acids which code for the rno proteins. The invention also provides nucleic acid molecules complementary to the nucleic acid molecules of SEQ ID NO: 1, SEQ ID NO: 3., and SEQ ID NO: 3.

The present invention also relates to an isolated nucleic acid having at least 15 consecutive nucleotides as represented by a nucleotide sequence selected from the nucleotides of rno-1, rno-2, and rno-3. A nucleotide having in the range from about 15 to about 30 consecutive nucleotides as represented by a nucleotide sequence selected from the nucleotides of rno-1, rno-2, and rno-3 is also within the scope of the present invention.

The present invention also provides recombinant vectors comprising nucleic acid molecules that code for a rno protein. These recombinant vectors may be plasmids. In other embodiments, these recombinant vectors are prokaryotic or eukaryotic expression vectors. The nucleic acid coding for a rno protein may also be operably linked to a heterologous promoter. The present invention further provides host cells comprising a nucleic acid that codes for a rno protein.

These and other advantages of the present invention will become apparent upon reading the following detailed description and appended claims.

### BRIEF DESCRIPTION OF THE FIGURES

In order that the manner in which the above-recited and other advantages and objects of the invention are obtained will be readily understood, a more particular description of the invention briefly described above will be rendered by reference to specific embodiments thereof which are illustrated in the appended figures. Understanding that these figures depict only typical embodiments of the invention and are not therefore to be considered to be limiting of its scope, the invention will be described and explained with additional specificity and detail through the use of the accompanying figures in which:

FIG. 1 is a northern blot showing the induction of rno expression by NO. HL-60 cells were treated for 24 hr with DETA-NO (125 μM). Total cellular RNA was isolated and northern analysis was performed by using radiolabelled rno DP-3A as a probe as shown in the upper panel. Analysis of the same blot by using a radio labelled GAPDH probe confirmed the integrity of the RNA and demonstrated equal loading of the lanes as shown in the lower panel. The positions of the bands representing 28S and 18S ribosomal RNA are indicated on the left. Lane 1, RNA from control, untreated cells; Lane 2, RNA from DETA-NO treated cells. DETA-NO upregulates rno expression in HL-60 cells.

FIG. 2 is a northern blot illustrating the tissue distribution of rno. Multiple tissue northern blots containing mRNA from normal and malignant human tissues were analyzed by using a radiolabelled rno probe (DP-3A). Lane 1, stomach; Lane 2, thyroid; Lane 3, spinal cord; Lane 4, lymph node; Lane 5, trachea; Lane 6, adrenal gland; Lane 7, bone marrow; Lane 8, spleen; Lane 9, thymus; Lane 10, prostate; Lane 11, testis; Lane 12, ovary; Lane 13, small intestine; Lane 14, colon; Lane 15, peripheral blood leukocyte; Lane 16, HL-60 cells (AML cell line); Lane 17, HeLa cells (fibroblast cell line); Lane 18, K562 cells (chronic myelogenous leukemia cell line); Lane 19, MOLT-4 cells (T lymphocyte cell line); Lane 20, Raji cells (B lymphocyte cell line); Lane 21, SW480 cells (colorectal cancer cell line) Lane 22, A549 cells (lung epithelial cancer cell line); Lane 23, G36 1 cells (melanoma cell line). The positions of the size standards in kilobases (kb) are indicated to the left of each blot. Constitutive expression of rno appears to be restricted to cells of haematopoietic origin. The only malignant cells analyzed that express rno are K562, a cell line derived from a patient with CML.

FIG. 3 is a northern blot illustrating rno expression in normal peripheral leukocytes. RNA was isolated from normal PMN (lane 1) and mononuclear cells (lane 2), and northern analysis was performed by using a radiolabelled rno probe (DP-3A) as shown in the upper panel. The positions of the bands representing 28S and 18S ribosomal RNA are indicated on the left. Analysis of the same blot using a radiolabelled GAPDH probe as shown in the lower panel confirmed the integrity of the RNA and demonstrated equal loading of the lanes. Compared to mononuclear cells, PMN express much greater amounts of rno. The length of exposure required to visualize adequately the rno band in the lane containing mononuclear cell RNA resulted in overexposure of the lane containing the PMN RNA.

FIG. 4 is a northern blot showing the effect of differentiating agents on rno expression in HL-60 cells. For the upper panel, HL-60 cells were treated with DETA-NO, DMSO, or VD₃ for 24 hours before analysis of rno expression by RT-PCR. Lane 1, no treatment control; Lane 2, DETA-NO; Lane 3, DMSO; Lane 4, VD3; Lane 5, water blank (no template); Lane 6, reverse transcriptase omitted from the reaction mixture; Lane 7, RT-PCR performed using RNA isolated from normal PMN (positive control). The positions of the size marker (MW) are indicated on the left. The positions of rno-1 and rno-3 are shown on the right. Referring to the lower panel, RT-PCR analysis of actin expression was used to control for the quality and integrity of the cDNA that was prepared from the various samples. Lane 1, no treatment control cells; Lane 2, cells treated with DETA-NO, Lane 3, cells treated with DMSO; Lane 4, cells treated with VD₃; Lane 5, water blank (no template), Lane 6, RT-PCR performed using RNA isolated from normal PMN. Expression of rno is upregulated by DETA-NO or DMSO but not by VD₃. rno-3 is identical to rno-1 except for a 171 bp deletion. The results of one experiment are shown; these results are representative of three experiments.

FIG. 5 illustrates the time course of rno induction in HL-60 cells. For the results of the upper panel, HL-60 cells were treated with 125 μM DETA-NO or 1.2% DMSO, and expression of rno was analyzed by RT-PCR at three different time points. MW, 100 bp DNA ladder; Lane 1, no-treatment control cells; Lane 2, cells treated with DETA-NO for 24 hr; Lane 3, cells treated with DETA-NO for 48 hr; Lane 4; cells treated with DETA-NO for 72 hr; Lane 5, cells treated with DMSO for 24 hr; Lane 6, cells treated with DMSO for 48 hr; Lane 7, cells treated with DMSO 72 hr; Lane 8, water blank (no template). The position of rno-1 is indicated on the left. For the results of the lower panel, RT-PCR analysis of actin expression was used to control for the quality and integrity of the cDNA that was prepared from the various samples. MW, 100 bp DNA ladder; Lane 1, no-treatment control cells; Lane 2, cells treated with DETA-NO for 24 hr; Lane 3, cells treated with DETA-NO for 48 hr; Lane 4; cells treated with DETA-NO for 72 hr; Lane 5, cells treated with DMO for 24 hr; Lane 6, cells treated with DMSO for 48 hr; Lane 7, cells treated with DMSO 72 hr. The position of actin is indicated on the left. DETA-NO upregulates rno within 24 hours and expression returns to baseline by 48 hours. DMSO also upregulates rno within 24 hours but expression persists through the entire 72 hr of exposure. The results of one experiment are shown; these results are representative of three experiments.

FIG. 6 illustrates the effect of cGMP on rno expression. In the upper pannel, HL-60 cells were treated with DETA-NO, 8Br-cGMP, ODQ, or ODQ and DETA-NO for 24 hours. Subsequently rno expression was analyzed by using RT-PCR. Lane 1, molecular mass standards (100 bp ladder); Lane 2, no-treatment control cells; Lane 3, DETA-NO; Lane 4, 8-Br-cGMP; Lane 5, ODQ; Lane 6, ODQ and DETA-NO; Lane 7, water blank (no cDNA template); Lane 8, reverse transcriptase omitted from one of the reaction mixtures. The position of rno-1 is indicated on the left. In the lower panel, RT-PCR analysis of actin expression was used to control for the quality and integrity of the cDNA that was prepared from the various samples. Lane 1,100 bp DNA ladder; Lane 2, no-treatment control; Lane 3, cells treated with DETA-NO; Lane 4, cells treated with 8Br-cGMP; Lane 5, cells treated with ODQ; Lane 6, cells treated with ODQ and DETA-NO. The position of actin is indicated on the left. Upregulation of rno by DETA-NO is cGMP-independent. The results of one experiment are shown; these results are representative of three experiments.

FIG. 7A is a schematic representation of rno and deduced amino acid sequence. The clone was isolated from a human leukocyte cDNA library. It is notable for a long (1308 bp) 5′ untranslated region (5′ UTR). The coding region of rno-1 is 1,032 nucleotides in length. The consecutive 171 bp deletions that define rno-2 and rno-3 are illustrated. The positions of the BglII sites are indicated. The DP-3A restriction fragment that was cloned using RDA was generated by BglII digestion of oligo dT primed cDNA. The 3′ untranslated region (3′ UTR) is also illustrated. This segment is 323 bp in length and contains both a polyadenylation signal and a polyA tail.

FIG. 7B shows the deduced sequence of rno-1 (SEQ ID NO: 2) using the single letter code for amino acids. The 57 amino acids (204-260) that are deleted in rno-2 (SEQ ID NO: 4) are indicated by the single underline while the 57 residues (261-317) that are deleted in rno-3 (SEQ ID NO: 6) are indicated by the double underline.

FIG. 8 shows a comparison of the primary sequence of RI (SEQ ID NO: 14) and rno (SEQ ID NO 1). Using the single letter code for amino acids, the deduced sequence of rno-1 was aligned with that of RI based on the pattern of leucine-rich repeats that characterize the primary structure of RI. Shared residues (denoted by a closed square) are shown in bold type. The single underlined residues denote the 57 amino acids that are deleted in rno-2 (SEQ ID NO: 3) and the double underlined residues denote the 57 amino acids that are deleted in rno-3 (SEQ ID NO 5). The numbers on either side of the rows of sequence indicate the amino acid position counting from the amino-terminus. On the far right, the designation A3, B3, etc. refers to the alternating A-type and B-type leucine-rich repeats of RI. Like RI, rno is composed of an alternating series of leucine-rich repeats. That the deletions in rno-2 and rno-3 are each 57 amino acids insures that the leucine-rich repeats will remain in-frame because the sum of the A repeat (28 amino acids)+the B repeat (29 amino acids) is 57.

FIG. 9 shows a comparison of the primary sequence of the A- and B-type repeats of rno-1. Using the single letter code for amino acids, the five A- and B-type repeats (SEQ ID NOS: 15-24) are aligned. Invariant residues are shown in bold type. Within each repeat, many of the residues are either invariant or highly conserved.

FIG. 10 is a series of graphs representing the effect of rno-2 expression on HL-60 cell growth: HL-60 cells were transiently transfected with the pcDNA3.1/rno-2 vector which expresses rno-2 under a CMV promoter. FIG. 10A illustrates a cell growth assay using a Coulter counter. FIG. 10B illustrates the number of apoptotic cells as determined by flow cytometry. Control mock transfections were done in parallel. The expression of rno-2 inhibited HL-60 cell growth and induces apoptosis in them. The drop in the proportion of apoptotic cells at day 3 is likely due to growth of non-transfected cells. The data illustrates averages and standard errors of the mean from 3 separate experiments.

FIG. 11 is a bar graph illustrating the effect of inducible expression of rno-2 on HL-60 cell growth:HL-60/TET and HL-60/TET/RNO2 cells were treated with 2 or 5 μg/ml Tetracycline to induce rno-2 expression. Seven days after the addition of Tetracycline, cell growth was assayed using a Coulter counter. Using an inducible expression system, rno-2 expression inhibited the growth of HL-60 cells. Growth inhibition was more pronounced with the higher dose of Tetracycline, suggesting that the growth inhibitory effects of rno-2 were dependant on the level of gene expression. Tetracycline did not affect cell growth in the absence of rno-2 expression (HL-60/TET cell line). Asterisks indicate statistically significant differences between treatment and controls. The data illustrate averages and standard errors of the mean of three separate experiments.

FIG. 12 are photographs of cells showing the effect of rno-2 expression on HL-60 cell differentiation:HL-60/TET/RNO2 cells were treated for 5 days with Tetracycline at a concentration of 5 μg/mL in order to induce rno-2 expression. Five days after Tetracycline addition, the cells were immobilized on a microscope slide by cytospin and stained with Wright's stain. FIG. 12 A shows untreated control cells. FIGS. 12B and 12C show Tetracycline treated cells. HL-60 cells expressing rno-2 developed the following features suggesting a monocytic phenotype: pale cytoplasm, decreased nuclear:cytoplasmic ratio, folded nucleus, and fine cytoplasmic granules. The results of one experiment are shown; these results are representative of three experiments.

FIG. 13 is a bar graph illustrating apoptosis induction in leukemia cells by rno-2 expression. In order to induce rno-2 expression, HL-60/TET/RNO2 cells were treated with a single dose of Tetracycline of 7.5 or 10 μg/mL. In separate experiments, HL-60/TET/RNO2 cells were treated with 7.5 μg/mL of Tetracycline daily for 3 days. Apoptosis was assessed by flow cytometry at day 3. Parallel control experiments were set-up with the same doses and schedule of Tetracycline using the parent HL-60 cell line. Tetracycline treatment did not significantly increase the number of apoptotic HL-60 cells. Tetracycline treatment (and therefore rno-2 expression) significantly increased the number of apoptotic HL-60/TET/RNO2 cells. Apoptosis induction was dose dependant suggesting that the effect of rno-2 is dependant on the level of expression. The averages and SEM of 3 separate experiments are shown. An asterisk denotes statistically significant differences between treatment and control.

FIG. 14 is a photograph showing the intracelluar distribution of rno-2. HL-60 cells were transfected with the pEGFP/RNO2 vector. Twenty four hours after transfection, cells were immobilized on a microscope slide using a cytospin. Expression of the fusion protein was evaluated by confocal microscopy. At 24 hours expressed rno-2 is present in the cytoplasm.

### DETAILED DESCRIPTION OF INVENTION

The present invention relates to a novel gene which is upregulated by NO (rno). More particularly, the present invention relates to the isolation and characterization of three isoforms of the rno gene, rno-1 (SEQ ID NO: 1), rno-2 (SEQ ID NO: 3), and rno-3 (SEQ ID NO: 5). Nucleotide sequences complementary to the nucleotide sequences of SEQ ID NO: 1, SEQ ID NO: 3, and SEQ ID NO: 5 are also provided. NO induces differentiation and inhibits growth of acute myeloid leukemia (AML) cells indicating that the rno gene products may have a role in the differentiation and apoptosis of AML cells.

Each of the isoforms of the rno gene code for a protein designated as rno-1 (SEQ ID NO: 2), rno-2 (SEQ ID NO: 3), and rno-3 (SEQ ID NO: 4). Isolated and purified nucleotide sequences that code for the amino acid sequence of the rno proteins are also within the scope of the invention.

The nucleic acid molecules that code for the rno can be contained within recombinant vectors such as plasmids, recombinant phages or viruses, transposons, cosmids, or artificial chromosomes. Such vectors can also include elements that control the replication and expression of the rno nucleic acid sequences. The vectors can also have sequences that allow for the screening or selection of cells containing the vector. Such screening or selection sequences can include antibiotic resistance genes. The recombinant vectors can be prokaryotic expression vectors or eukaryotic expression vectors. The nucleic acid coding for rno gene product can be linked to a heterologous promoter.

Host cells comprising a nucleic acid that codes for a rno gene product are also provided. The host cells can be prepared by transfecting an appropriate nucleic acid into a cell using transfection techniques that are known in the art. These techniques include calcium phosphate co-precipitation, microinjection, electroporation, liposome-mediated gene transfer, and high velocity microprojectiles.

A method for inducing the differentiation and inhibiting the growth of leukemia and other cancer cells is also provided. The method includes the step of contacting a cancer cell with a rno gene product. It has been shown that the insertion and expression of a rno gene into a cancer cell causes the differentiation and death of the cell. Thus, contacting a cell with an rno gene product may produce the same results.

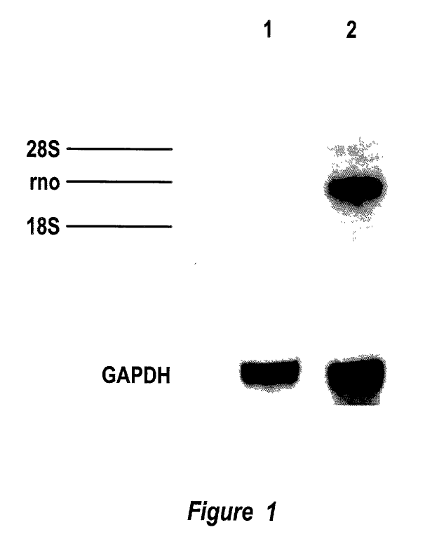

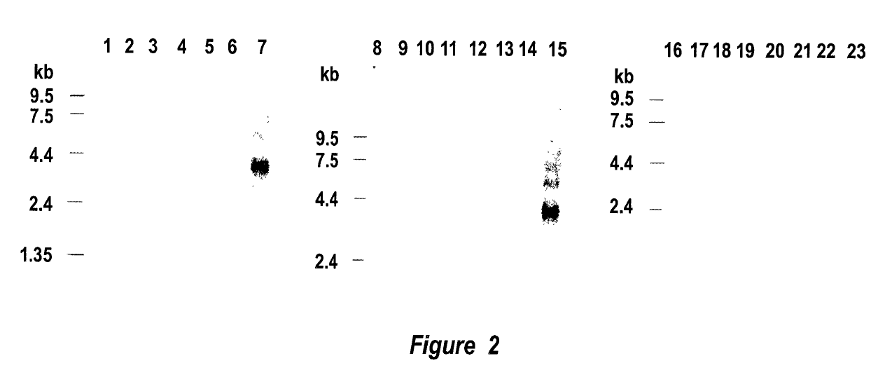

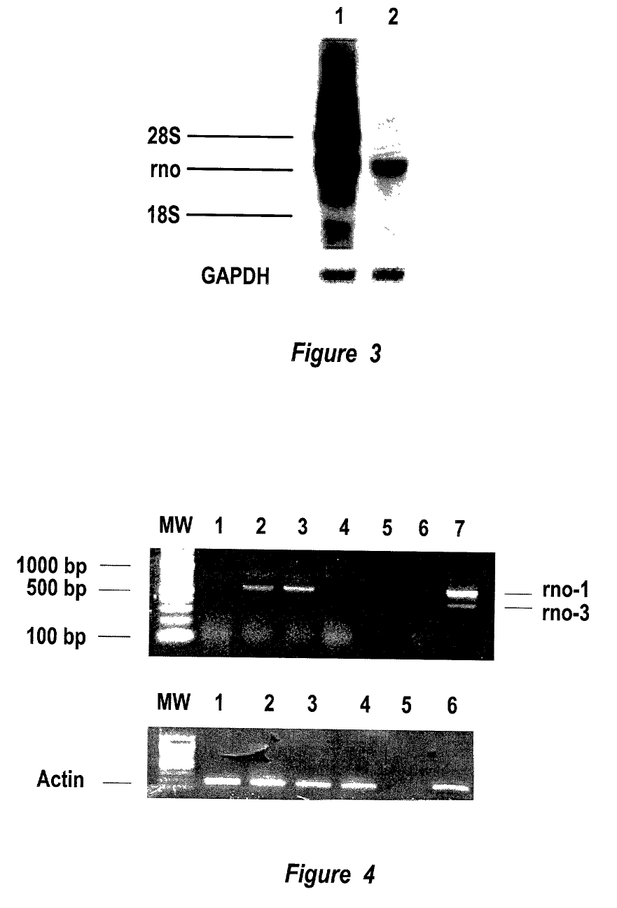

Referring to FIG. 1, genes involved in differentiation and growth inhibition of leukemia cells by NO were discovered by using RDA. A novel gene (rno) whose expression is induced by NO in HL-60 cells was discovered. The northern analysis illustrated in FIG. 2, suggests that constitutive expression of the rno is restricted to normal haematopoietic tissues. PMN are the primary source of rno in the peripheral blood, however, MN cells also express the gene constitutively as shown in FIG. 3. The highly restricted pattern of tissue distribution as shown in FIG. 2 suggests that rno has a specialized function in haematopoietic cells.

Referring to FIG. 4, rno expression is upregulated by both NO and DMSO but not by either VD3 or ATRA (data for ATRA not shown). DMSO and ATRA induce differentiation of HL-60 cells along granulocytic lines, and NO and VD₃ induce differentiation along monocytic lines. Magrinat, G et al. (1992) Blood, 80:1980-1986; Trayner et al. supra. Thus, these observations suggest that upregulation of rno expression is reagent specific rather than lineage specific. Previous studies have shown that NO induces differentiation of AML cells; however, the role of rno in this process is unknown. Magrinat, G et al. supra. Rather than being an integral part of the mechanism by which NO induces differentiation, upregulation of rno may represent a marker of the process. Study of rno expression in fresh AML isolates could yield valuable information on its role in haematopoietic cell growth and differentiation.

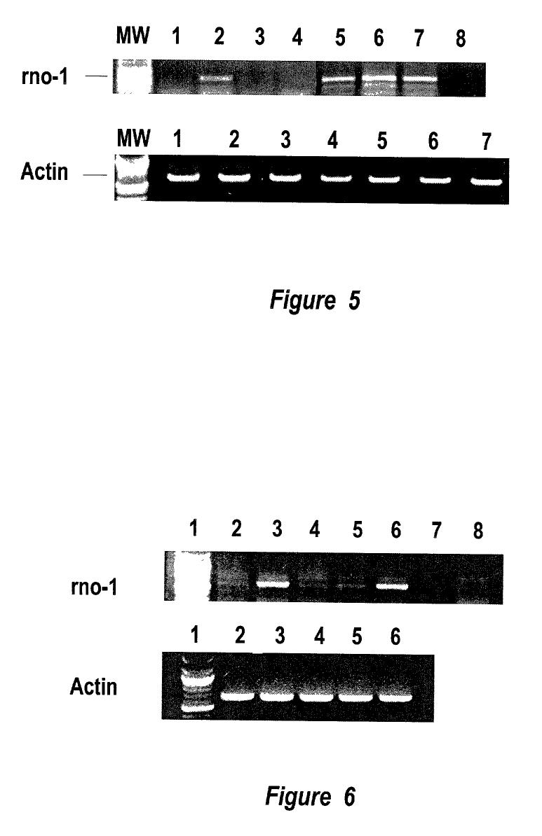

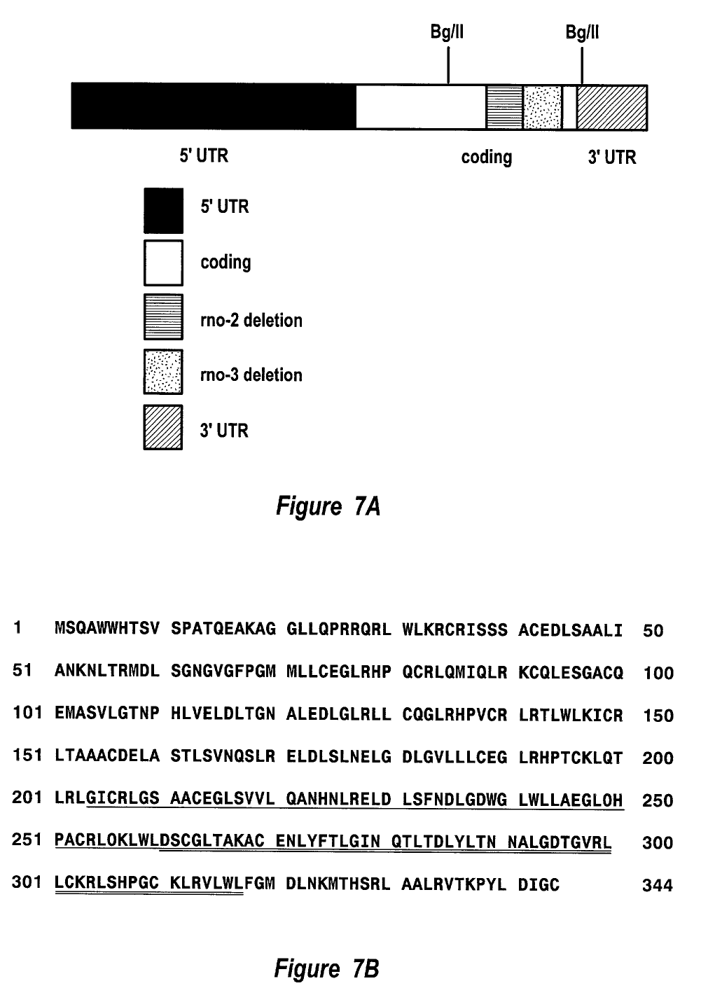

The mechanism whereby rno expression is upregulated by NO and DMSO is not presently known. However, as shown in FIG. 6, rno upregulation by NO is sGC/cGMP independent. One mechanism by which NO affects gene expression is oxidation of the iron-sulfur cluster of iron-regulatory protein-i (IRP-1). Drapier, J C et al. (1993) EMBO J.12(9):3643-3649; Weiss, Get al. (1993) EMBO J.12(9):3651-3657. In this case, loss of the iron-sulfur cluster enhances the affinity of IRP-1 for the iron responsive elements (IRE) found in the mRNA of H-ferritin, the transferrin receptor, and erythroid amino levulinate synthase (eALAS). Consequently, NO influences both the stability of transferrin receptor mRNA and the rate of translation of H-ferritin and eALAS mRNA. Although rno does not contain an IRE, the extremely long 5′ UTR is at least 1326 bp as shown in FIG. 7A. The 5′ UTR maybe the target of other trans-acting elements that are regulated by NO in a manner analogous to IRP-1. Both NO and DMSO modulate the cellular redox state. Wink, D A et al. (1999) Methods Enzymol.301:413-424; Joshi, M S et al. (1999) Free Radic Biol Med., 27(11-12):1357-1366; Kelly, K A et al. (1994) Infect Immun 62:3122-3128. That rno gene expression is enhanced by DMSO as well as NO supports the hypothesis that altering the redox state of target substrates may mediate rno upregulation by these reagents. Referring now to FIG. 5, time-course analysis suggests that the induction period for upregulation of rno by NO and DMSO is similar, but the effects of DMSO appear more sustained. The differentiating agents were added only once at the time of culture initiation. Because the half-life of DETA-NO is 20 hr, the functional activity of the reagent would have decreased by more than 75% by the time the 48 hr sample was harvested. It is likely that continuous upregulation of rno expression by NO requires a sustained concentration of the molecule. On the other hand, the chemical nature of DMSO makes it unlikely that a time dependent decay in activity would be observed.

At least three different rno isoforms exist. As seen in FIG. 7A, two of the isoforms, rno-2 and rno-3, appear identical to rno-1 except for deletions of 171 bp. The deletions involve consecutive runs of sequence within the coding region. The deletions, however, do not alter the reading frame. Consequently, the predicted amino acid sequence of rno-2 and 1 rno-3 is the same as that of rno-1 except for deletions of 57 residues that occur consecutively as seen in FIG. 7B. Conceivably, other isoforms of rno exist.

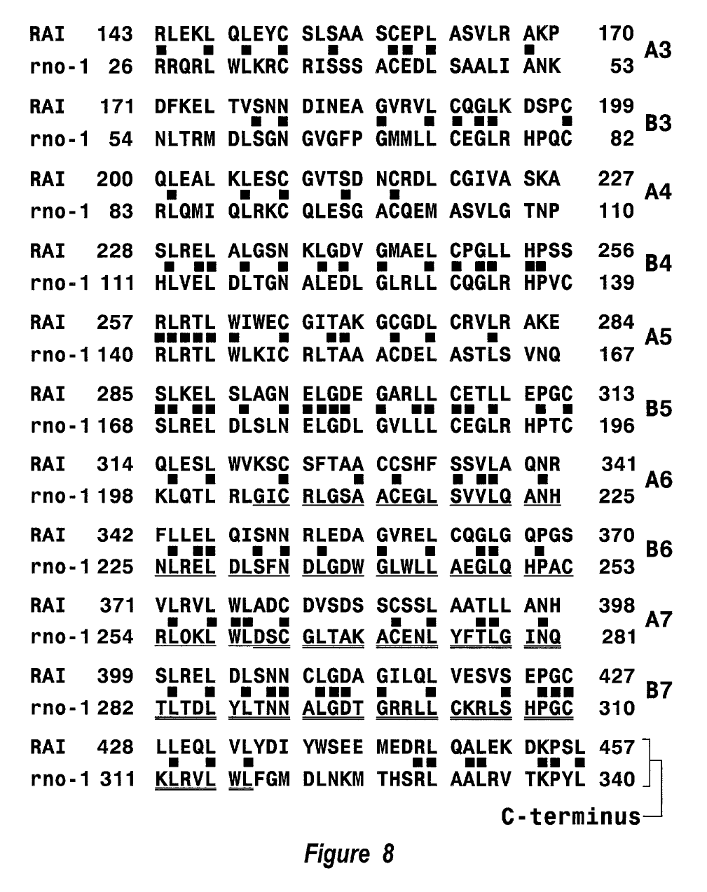

The deduced amino acid sequence of rno-I revealed a basic protein (estimated pI=9.68) that is leucine-rich and contains 20 cysteines. Significant homology with RI, the ribonuclease/angiogenin inhibitor was noted, and particularly striking was the conservation of the leucine and cysteine residues within the linear sequence as shown in FIG. 8. In addition, like RI, RNO-1 is comprised of a series of alternating A- and B-type LRR as shown in FIGS. 8 amd The A-type repeats consist of 28 amino acids and the B-type repeats consist of 29 residues (A+B =57 amino acids). The importance of the repeating structure is suggested by the observation that the RNO-2 and RNO-3 isoforms result from 57 amino acid deletions that preserve the organization of the LRR as shown in FIG. 8. In the case of RI, the alternating repeats play a critical role in determining the three dimensional structure of the protein. Hofsteenge, J. (1997) Ribonucleases: structure and functions, Academic Press 621-658, San Diego, Calif. Based on the homology of the primary structure, it is tempting to speculate that rno and RI also share functional properties. However, LRR have been identified in proteins with a diverse range of activity including enzyme inhibition or activation, DNA repair, RNA processing, signal transduction, and extracellular matrix interactions. Kobe, B & Deisenhofer, J. (1994) Trends Biochem Sci.19:415-421; Kobe, B & Deisenhofer, J (1995) Curr Opin Struct Biol.5:409-416. Thus, while rno and RI are structurally related, they may be functionally different. Support for this interpretation comes from the observation that the contact sites of RI for one of its primary substrates (RNAse A) are not conserved in rno. Hofsteenge, J. (1997) Ribonucleases: structure and functions.Academic Press 621-658, San Diego, Calif. All proteins that contain LRR appear to be involved in macromolecular interactions, suggesting that the repeat motif provides a versatile framework onto which specialized functions can be grafted. Kobe, B & Deisenhofer, J. (1994) Trends Biochem Sci.19:415-421; Kobe, B & Deisenhofer, J (1995) Curr Opin Struct Biol.5:409-416. The unique tissue distribution and structure of rno suggest a role in haematopoietic cell growth, differentiation or function.

In order to better describe the details of the present invention, the following discussion is divided into seven sections: (1) identification of rno; (2) tissue distribution of rno; (3) effects of differentiating agents on rno expression; (4) putative isoform of rno; (5) time course of rno induction; (6) effect of sGC/cGMP on rno expression; and (7) molecular cloning of the rno cDNA.

6.1 Identification of rno

The following two sets of cells were used for analysis of the effects NO on gene expression: control HL-60 cells incubated in media for 24 hours and HL-60 cells treated with 125 μM DETA-NO (a NO generating compound) for the same time period. Poly (A⁺) RNA was prepared from the two sets of cells, and this material was used as template for the synthesis of double stranded cDNA. Following digestion with BglII, a set of primer/adapters was ligated to aliquots of each sample, and this cDNA was amplified by PCR to generate representations from the control and treated populations. By using the technique of RDA modified for cDNA, gene expression between the control and DETA-NO treated cells was compared. Lisitsyn, N et al. (1993) Science.259:946-951; Hubank, M & Schatz, D G (1994) Nucl Acids Res. 22:5640-5648; Kanai, N et al. (1999) Am J Hematol., 61:221-231. In the present study, two sets of subtraction/hybridizations were performed in parallel. In set A, PCR amplified cDNA from the untreated control cells was used as driver and amplified cDNA from DETA-NO-treated cells was used as the tester. In set B, the subtraction/hybridization was performed in the opposite direction (i. e. amplified cDNA prepared from the DETA-NO-treated cells was used as driver, and amplified cDNA from control HL-60 cells was used as the tester). Three rounds of subtractive/hybridization were performed. In the first round the driver:tester ratio was 100:1; in the second round, the ratio was 800:1; and in the third round, the ratio was 400,000:1.

When, the PCR amplified difference products from the third round of subtractive/hybridization (DP-3) were analyzed by agarose gel electrophoresis and ethidium bromide staining, no bands were observed in the lane containing an aliquot of the PCR reaction from set B in which the control sample served as the tester (DP-3B). However, a band of 670 bp was observed in the lane containing DP-3A in which the DETA-NO-treated sample served as the tester. These results suggested the existence of a NO-inducible gene. To challenge this interpretation, the PCR product from set A (DP-3A) was directly cloned and used as a probe for northern analysis of RNA prepared from control and DETA-NO treated HL-60 cells. The results of the northernanalysis are shown in FIG. 1. The autoradiograph showed a single band of approximately 3.0 kb in the lane containing RNA from the DETA-NO treated cells as seen in FIG. 1, lane 2, but a corresponding band was not seen in the lane containing RNA from the untreated controls as seen in FIG. 1, lane 1. These results confirm that DP-3A is a true difference product representing a restriction fragment derived from a gene that is induced by NO.

Nucleotide sequencing using primers that flanked the cloning site of plasmid containing DP-3A revealed a 672 bp insert. The size of the BglII-BglII fragment was 624 bp (determined by subtracting the two 24 bp primer/adapters that were ligated to each end of the cDNA as part of the RDA procedure). When the nucleotide sequence of the BglII-BglII fragment was analyzed by BLASTN against non-redundant GenBank+EMBL+DDBJ +PDB identity with a known gene was not observed. Because expression was regulated by NO, the novel gene was named rno (regulated by nitric oxide). Further analysis of the nucleotide sequence showed that a 485 bp segment of DP-3A shared 54% identity with a similar sized segment of the 3′ end of mRNA for the human ribonuclease/angiogenin inhibitor (RI). Hofsteenge, J. (1997) Ribonucleases: structure and functions. Academic Press 621-658, San Diego, Calif.

6.2 Tissue Distribution of rno

Referring to FIG. 2, northern analysis using radiolabelled DP-3A as a probe showed that expression of rno was confined to haematopoietic tissues. The most intense band was observed in lane 15 which contained RNA from peripheral blood leukocytes. A distinct band was also seen in lane 7 which contained RNA from bone marrow cells. In both cases, three less intense bands were also visible. Northern analysis of RNA from cell-lines derived from malignant tissues of human origin showed that K562 cells (a chronic myelogenous leukemia line) expressed rno weakly as shown in FIG. 2, lane 18. These results suggest that expression of rno by malignant cells of either haematopoietic or non-haematopoietic origin is uncommon.

Referring to FIG. 3, peripheral blood leukocytes from a volunteer donor were separated into PMN and MN cell fractions for comparison of rno expression by northern analysis. In lane 2 which contained RNA from the MN cells, a single band of 3.0 kb was observed. PMN cells were found to express much higher levels of rno than MN cells. Furthermore, in addition to a 3.0 kb product expressed in both cell fractions, PMN cells express 3 larger products as shown in FIG. 3, lane 1. These results suggest that PMN are the source of most of the rno expressed by peripheral blood leukocytes as seen in FIG. 2, lane 15.

6.3 Effects of Differentiating Agents on rno Expression

Referring to FIG. 4, RT-PCR was used to compare the effects of three differentiating agents on expression of rno. NO induces HL-60 cells to differentiate along the monocytic lineage. Magrinat, G et al. (1992) Blood, 80:1980-1986. DMSO is a granulocytic differentiation agent, while VD₃ induces monocytic differentiation. Trayner, ID et al. (1998) Leuk Res., 22:537-547. Treatment of HL-60 cells with either NO or DMSO induced expression of rno as seen in FIG. 4, lanes 2 and 3, but treatment with VD3 did not as seen in FIG. 4, lane 4. In separate experiments that used the same design, ATRA [another inducer of granulocytic differentiation in HL-60 cells (Id.)] was found to have no effect on rno expression even after 72 hr of exposure (not shown).

6.4 Putative Isoform of rno

Because peripheral blood PMN express relatively large amounts of rno, RNA from PMN cells was used as a positive control in the RT-PCR experiments shown in FIG. 4. In lane 7 of FIG. 4 which contained the PMN RT-PCR product, 2 bands of different intensity were observed. Southern analysis demonstrated that both the upper and lower band hybridized specifically with radiolabelled DP-3A probe (not shown). Southern analysis also revealed that the lower band was present in the lanes containing the RT-PCR products generated from DETA-NO and DMSO-treated HL-60 cells (not shown). To analyze further the relationship between the upper and lower bands, the PMN RT-PCR products were directly cloned. Sequence analysis showed that the upper band consisted of 557 bp that were identical to sequence contained in DP-3A except for a single nucleotide substitution. The lower band consisted of 386 residues and was identical to the upper band except for a 171 bp deletion (not shown). Thus, the lower band appears to represent an alternatively spliced form of rno.

6.5 Time Course of rno Induction

Referring to FIG. 4, both NO and DMSO induce expression of rno. To determine if the time-course of induction is similar, HL-60 cells were incubated with either DETA-NO or DMSO for 72 hr. The differentiating agent was added only at the beginning of the incubation. At timed intervals (24 hr, 48 hr, and 72 hr) cells were harvested and RNA was prepared for RT-PCR analysis of rno expression. Induction of rno expression by DETA-NO was evident at 24 hr and returned to baseline by 48 hr as shown by FIG. 5. DMSO also induced expression of rno within 24 hr, however, expression persisted through the entire 72 hr observation period. These results suggest that the effects of DMSO on rno expression are more sustained than those of DETA-NO.

6.6 Effect of sGC/cGMP on rno Expression

Referring to FIG. 6, many of the biological effects of NO are mediated by direct activation of the soluble guanylate cyclase (sGC)/cGMP signal transduction pathway. Hobbs, A J & Ignarro, L J (1996) Methods Enzymol.269:134-138. To determine if upregulation of rno is mediated by NO activation of sGC, HL-60 cells were treated with DETA-NO in combination with the sGC inhibitor ODQ. ODQ alone did not upregulate rno expression as seen in FIG. 6, lane 5. ODQ alone also did not prevent the induction of rno by DETA-NO as shown in FIG. 6, lane 6. Furthermore, the membrane permeable cGMP analogue 8-Br-9 cGMP did not induce rno expression as seen in FIG. 6, lane 4. These results indicate that the effects of NO on rno expression are sGC/cGMP independent.

6.7 Molecular Cloning of the rno cDNA

Referring to FIG. 7A, by screening a normal human peripheral blood leukocyte cDNA library, a clone that hybridized specifically with radiolabelled DP-3A was isolated. Nucleotide sequencing revealed an insert size of 2494 bp. Comparison of the sequence with that of the BglII-BglII fragment of DP-3A showed two single nucleotide substitutions and a 171 bp deletion. The deleted segment involved the nucleotides immediately upstream to those deleted in the lower band derived from RT- PCR amplification of neutrophil RNA. The sequence of the rno clone has been entered into the GenBank database (accession #AF231021).

Referring to FIG. 7B, the cloned cDNA had an open reading frame of 861 nucleotides (from residue 1309 to residue 2169) that encoded 287 amino acids. Thus, rno appears to have an extremely long 5′ untranslated region consisting of at least 1308 bp as shown in FIG. 7A. Nucleotides -5, -4, and -3 upstream from the AUG initiation codon conformed to the consensus sequence for eukaryotic initiation sites as described by Kozak. Lisitsyn, N et al. (1993) Science.259:946-951. Because this isoform has a 171 bp deletion, it was designated rno-2 to distinguish it from the putative full-length isoform (designated rno-1) that was identified by RDA and by RT-PCR of PMN RNA as seen in FIG. 4, lane 7. The isoform with the 171 bp deletion involving the sequence immediately downstream from the rno-2 deletion that was identified by sequencing the PMN RT-PCR product was named rno-3. When the 171 bp that comprise the rno-3 deletion were removed from rno-1, the coding region remained in-frame. Thus, comparison of the predicted sequence of the rno isoforms shows that the 57 amino acid deletions in rno-2 and rno-3 are consecutive and non-overlapping. Analysis of the deduced amino acid sequence of rno-2 and rno-3 revealed that both proteins have basic pI's, are leucine/cysteine rich, and have a calculated mass of 32 kD.

To complete the sequence that putatively corresponds to rno-1, the larger, predominant RNA species seen in FIG. 4, lane 7, the 171 bp that were deleted in rno-2 were reinserted into the leukocyte-derived cDNA clone. This addition, generated a 2683 bp polynucelotide. The derived clone contained only 2 BglII restriction sites (at positions 1766 and 2389, numbered from the 5′ end of the expanded clone). The 2 restriction sites were located in relative proximity to the poly A tail (the consensus polyadenylation signal AATAAA (SEQ ID NO: 11) is located beginning at position 2647 and the poly A tail starts at position 2668). Except for the same 2 nucleotide substitutions noted above, the predicted restriction fragment generated by cleavage of the derived clone with BglII is identical to DP-3A, thus accounting for the results of the RDA. When the sequence of the entire derived polynucleotide was compared to the non-redundant GenBank human EST database, almost complete identity with a 509 bp entry (accession # AA514928) was revealed (508 of 509 nucleotides were identical, 99.8%).

Three differences in the nucleotide sequence were observed when comparisons were made among the clone isolated from the leukocyte cDNA library, DP-3A, the neutrophil PCR products, and EST AA514928. Assuming that the rno sequence derived from the leukocyte cDNA library is correct, these variants are most likely PCR related artifacts in the case of DP-3A and the neutrophil PCR products, and a sequencing error in the case of EST AA514928. The possibility that sequence variants or acquired mutations account for the differences, however, cannot be excluded with certainty.

Referring to FIG. 8, when the deleted 171 bp was inserted at the appropriate site in the leukocyte derived cDNA clone, the coding region remained in-frame (open reading frame of 1,032 nucleotides between residues 1327-2358). The derived amino acid sequence of rno-1 predicted a protein consisting of 344 residues with a calculated Mᵣ of 38,060 D and an estimated pI of 9.68. The derived protein is leucine-rich (21%, 74/344 residues) and contains 20 cysteines. When the primary sequence was compared to the NCBI database (GenBank CDS translations+PDB+SwissProt+SPupdate+PIR) significant homology [identity =123/319 (38%), positives =183/319 (57%)] was observed with a 319 residue region of the human ribonuclease/angiogenin inhibitor (RI). Residues 141-459 of RI were aligned with residues 24-342 of rno-1. The positions of 49 of the 72 leucines (68%) of the deduced protein were conserved [the positions of 49 of 59 (83%) of the leucines of RI were conserved]. The positions of the cysteines were also highly conserved [16 of the 19 cysteines (84%) of rno-I aligned with RI, while 80% (16/20) of the RI cysteine positions aligned with rno-1].

Analysis of the deduced primary structure of rno-I showed that it is comprised of a series of leucine-rich repeats (LRR) similar to those of RI. LRR vary in length from 20-29 amino acids and contain leucines or other aliphatic residues at positions 2, 5, 7, 12, 16, 21, and 24, and asparagine, cysteine, or threonine at position 10. Kobe, B & Deisenhofer, J. (1994) Trends Biochem Sci.19:415-421; Kobe, B & Deisenhofer, J (1995) Curr Opin Struct Biol.5:409-416. RI consists of a series of alternating LRR. Hofsteenge, J. (1997) Ribonucleases: structure and functions, Academic Press 621-658, San Diego, Calif. The A-type repeat of RI is 28 amino acids in length. It has cysteine at position 10 with the consensus sequence CXLTXXXC (SEQ ID NO: 12) occupying residues 10-17. The B-type repeat is 29 amino acids in length. It has asparagine at position 10 with the consensus sequence NXLGDXG (SEQ ID NO: 13) at positions 10-16. Beginning with A3 in RI, the alternating A- and B-type LRR that characterize the primary structure of RI are also found in rno-1. Because the A-type repeat is 28 amino acids in length and the B-type repeat is 29 amino acids long, the pattern is repeated every 57 residues. Thus, the 57 amino acid deletions in rno-2 and rno-3 do not alter the repeat structure because the deletion is the same length as the combination of one type A (28 amino acids) and one type B (29 amino acids) repeat. Consequently, the LRR structure is preserved in both rno-2 and rno-3.

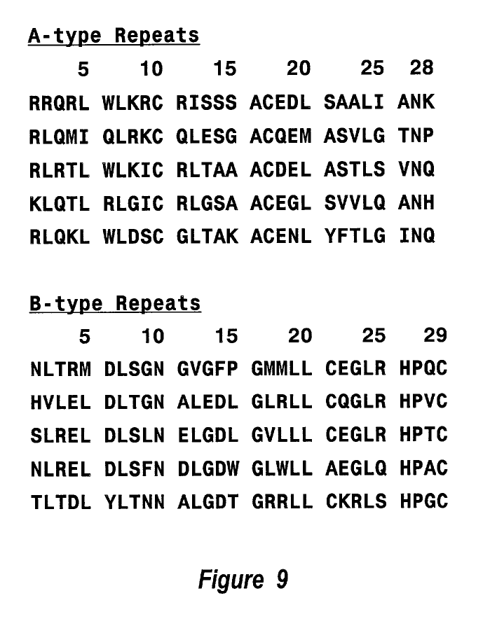

Referring to FIG. 9, among the A-type repeats of rno-1, 5 of the 28 residues are invariant, and at six other positions, 4 of the 5 residues are identical. The primary structure of the B-type repeats is more highly conserved, however, with 10 of the 29 amino acids being invariant and eight other positions having 4 of 5 residues identical. The conservation is particularly striking in the second half of the repeat where 7 of the last 14 residues are invariant.

All patents, publications, and commercial materials cited herein are hereby incorporated by reference.

### 7. EXAMPLES

The following examples are given to illustrate various embodiments which have been made with the present invention. It is to be understood that the following examples are not comprehensive or exhaustive of the many types of embodiments which can be prepared in accordance with the present invention.

### Example 1

### Cell Line and Culture Conditions

HL-60 cells were from ATCC (Rockville, Md.) and were cultured at 37° C. in a 5% CO₂ humidified atmosphere at a density of 150,000 cells/ml. The culture medium consisted of RPMI-1640 supplemented with 10% fetal bovine serum and 1% Penicillin (10,000 U/mL)/Streptomycin (10 mg/mL). (Z)-1-[2-(2-Aminoethyl)-N-(2-ammonioethyl)amino]diazen-1-um-1,2-diolate] (DETA-NO) was used as a source of NO. DETA-NO belongs to the diazenium diolate class of NO donors and releases 2 molecules of NO per carrier moiety with a half-life of NO release of 20 hours. Keefer, L K et al. (1996) Methods in Enzymology.268:281-293. The effects of the following other differentiating agents on expression of rno were also investigated: dimethyl sulfoxide (DMSO); all-trans retinoic acid (ATRA); and vitamin D₃ (VD₃). DETA-NO, DMSO, ATRA, or VD₃ were added once to HL-60 cells at the time of culture initiation at concentrations of 125 μM, 1.2% (v/v), 1 μM, and 100 nM, respectively. For studies of the role of the soluble guanylate cyclase (sGC)/cGMP signal transduction pathway in NO upregulation of rno, the sGC inhibitor 1H-[1,2,4]oxadiazolo[4,3-a]quinoxalin-1 -one (ODQ) and the cell permeable cGMP analogue 8-Br-cGMP at concentrations of 10 μM were used and 3 mM, respectively. Brunner, F et al. (1996) J Pharmacol Exp Ther. 277:48-53; Boss, GR.(1989) Proc Natl Acad Sci USA. 86:7174-7178. DETA-NO and ODQ were from Alexis (San Diego, Calif.). DMSO, ATRA, VD₃, and 8-Br-cGMP were from Sigma (St. Louis, Mo.).

### Example 2

### Peripheral Blood Leukocyte Isolation

After obtaining informed consent, 10 ml of heparinized peripheral blood were phlebotomized from a normal donor. Mononuclear cells were separated from polymorphonuclear leukocytes (PMN) by using a combination of dextran sedimentation and gradient density centrifugation on Ficoll Hypaque. Coligan, J E et al. (1999) Current Protocols in Immunology, John Wiley and Sons, Inc., New York, N.Y. Total cellular RNA was isolated with the QIAGEN RNeasy Mini Kit (QIAGEN, Santa Clara, Calif.) using the manufacturer's protocol. The samples were used for northern or reverse transcriptase polymerase chain reaction analysis. Blots were initially analyzed by using a radiolabelled rno probe generated by RDA (described in detail below). To assess the quantity and integrity of the RNA used in these experiments, the radioactivity was eluted using a heated solution of 0.25% SDS, and the blots were subsequently reanalyzed using a radiolabelled glyceraldehyde phosphate dehydrogenase (GAPDH) probe.

### Example 3

### Representational Difference Analysis (RDA)

The method was developed originally by Lisitsyn and colleagues for analysis of genomic DNA. Lisitsyn, N et al. (1993) Science., 259:946-951. To compare patterns of gene expression, the a modified procedure was used. Hubank, M & Schatz, D G (1994) Nucl Acids Res., 22:5640-5648. In the present study, minor modifications of that method were used as previously described. Kanai, N et al. (1999) Am J Hematol., 61:221-231. RDA is a PCR-based technique that is similar conceptually to other subtractive methods in that one cDNA population (the driver) is hybridized in excess against a second population (the tester) to remove common sequences, thereby enriching for sequences unique to the tester.

### Example 4

### Cloning of Difference Products

After three rounds of hybridization and subtraction, difference product 3 from set A (DP-3A) was directly cloned using the Original TA Cloning Kit (Invitrogen Corp, San Diego, Calif.). Plasmid DNA was isolated by using the Qiaprep Spin Plasmid Kit (QIAGEN, Santa Clara, Calif.). Following excision using EcoRI (New England Biolabs, Beverly, Mass.), the size of each cloned insert was estimated by agarose gel electrophoresis and ethidium bromide staining. Nucleotide sequencing of the appropriate size insert was performed by the University of Utah Health Sciences Center Sequencing Facility. Sequence homology searches were conducted using MacVector 5.0 software (Oxford Molecular Group, Campbell, Calif.). Nucleotide sequences were compared with the NCBI databases by using the Basic Local Alignment Search Tool Nucleotide (BLASTN) program. The deduced amino acid sequence of rno was compared to the NCBI databases by using the Basic Local Alignment Search Tool Protein (BLASTP) program.

### Example 5

### Northern Blot Analysis

Total cellular RNA from control or DETA-NO-treated cells was electrophoresed in a 1.2% denaturing agarose gel and transferred to a nylon membrane. Sambrook, J et al. (1989) Molecular cloning, 2^{nd} Ed, Cold Spring Harbor Laboratory Press. The blots were probed with radiolabelled DP-3A by using the QuickHyb solution system (Invitrogen Corp., San Diego, Calif.) according to the manufacturer's recommendations. In order to determine the tissue distribution of rno, Multiple Tissue Northern Blots from CLONTECH (Palo Alto, Calif.) were used with radiolabelled DP-3A as the probe.

### Example 6

### Reverse Transcriptase Polymerase Chain Reaction (RT-PCR)

The RT-PCR kit from Perkin-Elmer (Foster City, Calif.) was used in these experiments. Total cellular RNA was reversed transcribed by using an oligo dT primer. PCR conditions were as follows: denature 95° C. for 5 min, followed by 35 cycles of 1 min at 94° C. (denaturation); 1 min at 55° C. (annealing); 1 min at 72° C. (extension). Amplification was followed by a final extension step at 72° C. for 7 min. PCR primers were as follows: rno upstream primer, 5′ gataagaccattcagcagccaatg 3′ (SEQ ID NO: 7); rno downstream primer, 5′ agtgtgaaccagagcctgagagag 3′ (SEQ ID NO: 8), actin upstream primer, 5′-cgctgcgctggtcgtcgaca 3′ (SEQ ID NO: 9); actin downstream primer, 5′ gtcacgcacgatttcccgct 3′ (SEQ ID NO: 10). PCR products were electrophoresed in a 1% agarose gel and stained with ethidium bromide. RT-PCR products were cloned directly using the Original TA Cloning Kit (Invitrogen Corp, San Diego, Calif.). The nucleotide sequence of the appropriate size inserts was determined by the Sequencing Facility of the University of Utah Health Sciences Center. Southern analysis of RT-PCR products was performed using standard methods. Sambrook, J et al. (1989) Molecular cloning, 2^{nd} Ed, Cold Spring Harbor Laboratory Press.

### Example 7

### Cloning of rno cDNA

To isolate a cDNA clone of rno, a normal peripheral blood leukocyte cDNA library was screened (Human Leukocyte 5′-STRETCH PLUS cDNA Library from CLONTECH, Palo Alto, Calif.). In this library, the cDNA is directionally cloned at the EcoRI/XbaI sites of the phagemid vector λTriplEx. The cloning sites are located within a plasmid (λTriplEx) which is embedded in a X phage genome. The plasmid is flanked by loxP sites. Consequently, transduction of recombinant phage into a bacterial host (in this case E. Coli BM25.8) expressing cre recombinase constitutively releases the λTriplEx plasmid making it accessible for analysis following propagation in E. coli. The library was screened by the plaque lysis method using radiolabelled DP-3A as the probe. After the second round of screening, a strongly positive clone was identified and isolated. The presence of rno sequence in the clone was confirmed by PCR. Next, the λTriplEx phagemid vector containing the positive insert was converted into λTriplEx plasmid by transduction into BM25.8 E. Coli. After propagation in the cre recombinase containing bacteria, the plasmid was isolated using the Qiaprep Spin Plasmid Kit (QIAGEN, Santa Clara, Calif.). Sequencing of the insert was performed by the core sequencing facility of the University of Utah using plasmid specific primers followed by primer walking until the full length of the clone was sequenced.

### Example 8

### Effect of rno-2 Expression on HL-60 Cell Growth

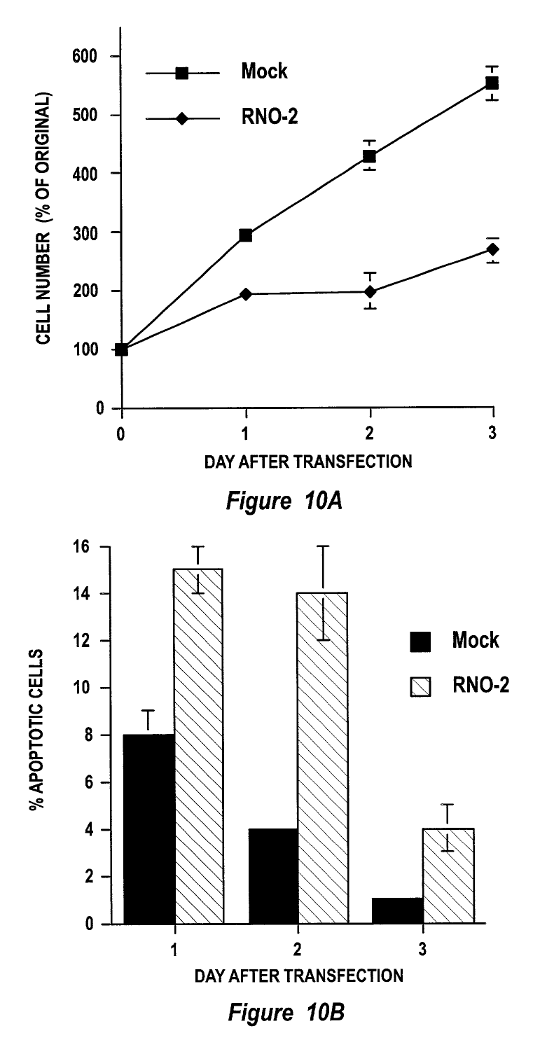

Expression vectors for the rno-2 isoform were made in order to determine the function of rno-2. In a first set of experiments, the cDNA for rno-2 was cloned in the pcDNA3.1 expression vector from Invitrogen (Carlsbad, Calif.). The expression vector obtained was named pcDNA3.1/rno-2. This vector allows the constitutive expression of the gene of interest under a CMV promoter. Transient transfection of HL-60 cells with pcDNA3.1/rno-2 lead to significant growth inhibition as compared to mock-transfected cells as shown in FIG. 10A. Referring to FIG. 10B, there was an increase in the number of apoptotic cells in pcDNA3.1/rno-2 transfected HL-60 cells as compared to mock-transfected cells 24 and 48 hours after transfection. Three days after transfection, the number of apoptotic cells went down in rno-2-transfected cells but remained higher than in mock-transfected cells. The latter observation is likely due to growth of non-transfected cells after 3 days since these were transient transfection experiments.

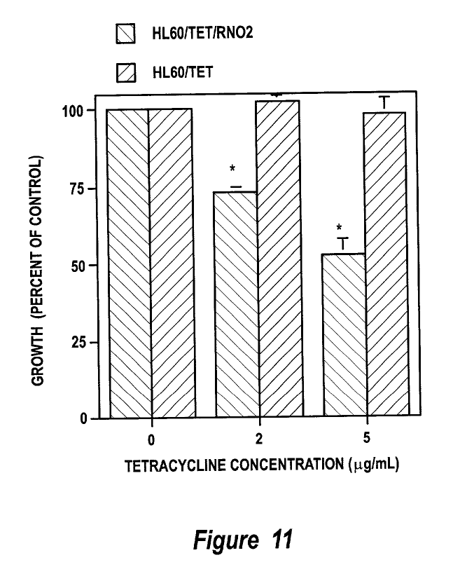

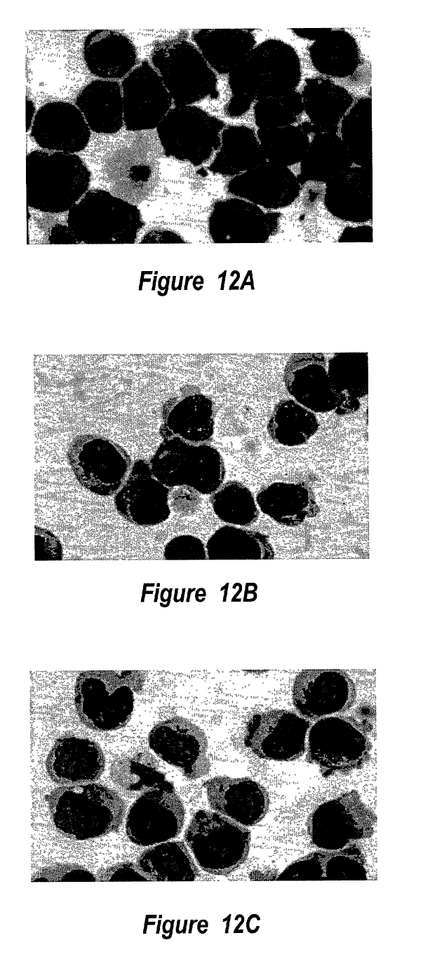

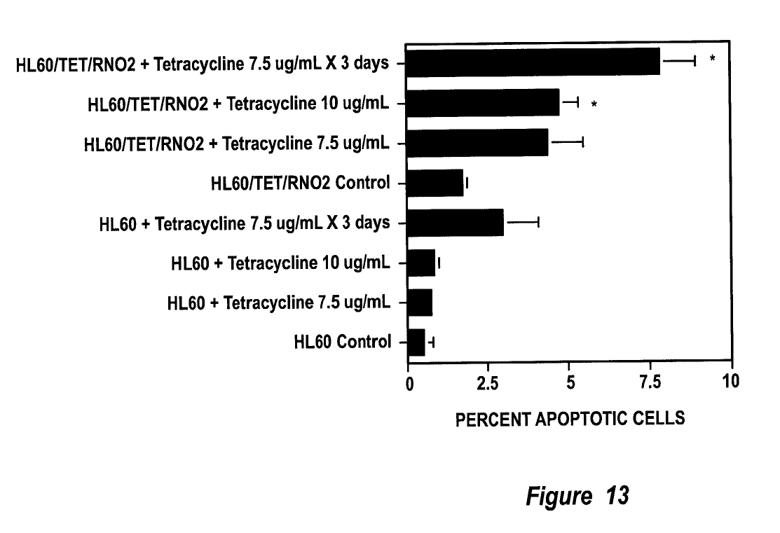

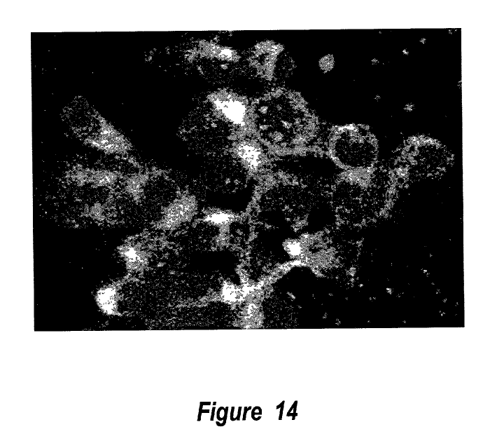

In separate experiments, the T-Rex inducible expression system from Invitrogen was used to make a stably transfected HL-60 cell line that expresses the Tetracycline Repressor Protein (TRP). The cell line was named HL-60/TET. This allows transfection of the cell line with expression vectors under the control of the Tetracycline Repressor Element (TRE), thus creating an inducible expression system. rno-2 was cloned in the pcDNA4/TO vector from Invitrogen. This vector was named pcDNA4/TO/rno-2 and contains the TRE which allows inducible expression of the gene of interest by treatment with Tetracycline. pcDNA4/TO/rno-2 was successfully transfected into HL-60/TET cells and obtained a stably double-transfected cell line that expresses rno-2 upon treatment with Tetracycline. That cell line was named HL-60/TET/RNO2. Tetracycline treatment of HL-60/TET/RNO2 cells lead to significant growth inhibition as shown in FIG. 11. Furthermore, there was a dose response relationship between the Tetracycline concentration and growth inhibition thus suggesting that the effect of rno-2 on cell growth is dependant on the level of gene expression. Importantly, control HL-60/TET cells that do not have the pcDNA4/TO/rno-2 vector (and therefore do not express rno-2 upon Tetracycline treatment) were not affected by Tetracycline. In another set of experiments, treatment of HL-60/TET/RNO2 cells with Tetracycline lead to morphologic changes suggestive of a monocytic phenotype as seen in FIG. 12. The cells expressing rno-2 had a lighter cytoplasm, folded nuclei, a lower nuclear:cytoplasmic ratio, and fine cytoplasmic granules. Further studies using the HL-60/TET/RNO2 cell line revealed that rno-2 expression induces apoptosis in those cells as seen in FIG. 13. These findings strongly suggest that rno expression affects leukemia cell growth and differentiation.

Using the pEGFP-N1 vector from CLONTECH, an expression system was made that expresses rno-2 fused to the N-terminus of the Enhanced Green Fluorescence Protein (EGFP discussed in detail in Aim 2). The vector has been named pEGFP/RNO-2. HL-60 cells were transfected with this fusion vector. Twenty four hours after transfection, confocal microscopy revealed that rno-2 was expressed as a cytoplasmic protein. Small quantities of rno-2 protein was used to immunize rabbits for antisera (not shown). These preliminary rno-2 expression experiments suggest that rno is a cytoplasmic protein that affects leukemia cell growth and differentiation. Furthermore, they demonstrate that rno expression systems in HL-60 cells can be made.

### SUMMARY

Nitric oxide (NO) inhibits growth and induces differentiation in acute myeloid leukemia (AML) cells. The effect of NO on AML gene expression was studied using the technique of Representational Difference Analysis. These data were used to identify genes associated with the growth inhibition and induction of differentiation. Exposure of HL-60 cells to the NO donor DETA-NO for 24 hours induced the expression of a novel gene that was named rno (regulated by nitric oxide). Treatment of HL-60 cells with DMSO induced expression of rno, but treatment with Vitamin D₃ or all-trans retinoic acid did not. Upregulation of rno by NO was cGMP-independent. Northern analysis indicated that constitutive expression of the novel gene was limited to leukocytes. Three isoforms of rno were identified. An rno cDNA clone was obtained by screening a human leukocyte library. The nucleotide sequence of the open reading frame shared significant homology with that of the human ribonuclease/angiogenin inhibitor (RI). The predicted amino acid sequence indicated that, like RI, rno is leucine and cysteine rich and is comprised of a series of repetitive elements (leucine-rich repeats) that may mediate macromolecular interactions. Expression of rno in HL60 cells induces growth arrest, differentiation, and apopotosis. Enhancement of expression of rno may be a component of the process by which differentiation and growth inhibition of leukemia cells is induced by NO.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

We claim:

1. An isolated nucleic acid molecule comprising a nucleotide sequence having at least 15 consecutive nucleotides as represented by a nucleotide sequence selected from the group consisting of nucleotides 1 through 2641 of SEQ ID NO: 1, nucleotides 1 through 2455 of SEQ ID NO: 3, and nucleotides 1 through 2471 of SEQ ID NO: 5.

2. An isolated and purified nucleic acid, the nucleic acid comprising nucleotides which code for the amino acid sequence of SEQ ID NO: 2.

3. A recombinant vector comprising the nucleic acid molecule of claim 2.

4. A host cell comprising the vector of claim 3.

5. An isolated and purified nucleic acid comprising the nucleotide sequence of SEQ ID NO: 1 or a nucleotide sequence complementary to the nucleotide sequence of SEQ ID NO: 1.

6. A recombinant vector comprising the nucleic acid molecule of claim 5.

7. A host cell comprising the vector of claim 7.

8. The host cell of claim 7, wherein the host cell is a eukaryotic host cell.

9. An isolated and purified nucleic acid, the nucleic acid comprising nucleotides which code for the amino acid sequence of SEQ ID NO: 4.

10. A recombinant vector comprising the nucleic acid molecule of claim 9.

11. The recombinant vector of claim 10, wherein the recombinant vector is a plasmid.

12. A host cell comprising the vector of claim 10.

13. The host cell of claim 12, wherein the host cell is a eukaryotic host cell.

14. An isolated and purified nucleic acid comprising the nucleotide sequence of SEQ ID NO: 3 or a nucleotide sequence complementary to the nucleotide sequence of SEQ ID NO: 3.

15. A recombinant vector comprising the nucleic acid molecule of claim 14.

16. The recombinant vector of claim 15, wherein the recombinant vector is a plasmid.

17. A host cell comprising the vector of claim 15.

18. The host cell of claim 17, wherein the host cell is a eukaryotic host cell.

19. An isolated and purified nucleic acid, the nucleic acid comprising nucleotides which code for the amino acid sequence of SEQ ID NO: 6.

20. A recombinant vector comprising the nucleic acid molecule of claim 19.

21. The recombinant vector of claim 20, wherein the recombinant vector is a plasmid.

22. A host cell comprising the vector of claim 20.

23. The host cell of claim 22, wherein the host cell is a eukaryotic host cell.

24. An isolated and purified nucleic acid comprising the nucleotide sequence of SEQ ID NO: 5 or a nucleotide sequence complementary to the nucleotide sequence of SEQ ID NO: 5.

25. A recombinant vector comprising the nucleic acid molecule of claim 24.

26. The recombinant vector of claim 25, wherein the recombinant vector is a plasmid.

27. A host cell comprising the vector of claim 25.

28. The host cell of claim 27, wherein the host cell is a eukaryotic host cell.

29. An isolated protein comprising an amino acid sequence selected from the group consisting of the amino acid sequence of SEQ ID NO: 2, the amino acid sequence of SEQ ID NO: 4, the amino acid sequence of SEQ ID NO: 6, an amino acid sequence that differs from the amino acid sequence of SEQ ID NO: 2 by one or more conservative amino acid substitutions only, an amino acid sequence that differs from the amino acid sequence of SEQ ID NO: 4 by one or more conservative amino acid substitutions only, an amino acid sequence that differs from the amino acid sequence of SEQ ID NO: 6 by one or more conservative amino acid substitutions only, an amino acid sequence having at least 80% identity to the complete amino acid sequence comprising SEQ ID NO: 2, an amino acid sequence having at least 80% identity to the complete amino acid sequence comprising SEQ ID NO: 4, and an amino acid sequence having at least 80% identity to the complete amino acid sequence comprising SEQ ID NO: 2.

30. An isolated protein comprising an amino acid sequence having at least 15 consecutive amino acid residues as represented by an amino acid sequence selected from the group consisting of the amino acid residues of SEQ ID NO: 2, the amino acid residues of SEQ ID NO: 4, and the amino acid residues of SEQ ID NO: 6.

31. A method of inducing differentiation and death in a cancer cell, the method comprising:
contacting the cancer cell with a molecule, the molecule comprising a protein comprising an amino acid sequence selected from the group consisting of the amino acid sequence of SEQ ID NO: 2, the amino acid sequence of SEQ ID NO: 4, and the amino acid sequence of SEQ ID NO: 6.

32. A method of diagnosing acute myeloid leukemia comprising: detecting the expression of a rno gene comprising nucleotides which code for an amino acid sequence selected from the group consisting of SEQ ID NO: 2, SEQ ID NO: 4, and SEQ ID NO: 6.
